(19) United States
(12) Patent Application Publication
(10) Pub. No.: US 2021/0396749 A1
(43) Pub. Date: Dec. 23, 2021

(54) DIAGNOSTIC TESTING DEVICE

(71) Applicants: Rick Hennessey, Pompano Beach, FL (US); Barry Abraham, Fort Lauderdale, FL (US)

(72) Inventors: Rick Hennessey, Pompano Beach, FL (US); Barry Abraham, Fort Lauderdale, FL (US)

(21) Appl. No.: 17/356,189

(22) Filed: Jun. 23, 2021

Related U.S. Application Data
(60) Provisional application No. 63/042,702, filed on Jun. 23, 2020.

(51) Int. Cl. G01N 33/558; B32B 7/12; B32B 5/18; B32B 37/12; B32B 37/20; B32B 38/00; B32B 38/18

(52) U.S. Cl. CPC G01N 33/558; B32B 7/12; B32B 5/18; C12Q 1/686; B32B 37/20; B32B 38/0004; B32B 38/1858; B32B 37/12

(57) ABSTRACT

A diagnostic testing device along with a method of manufacturing the diagnostic testing device is provided. The diagnostic testing device includes a sheet of foam; a top web including a recess shaped and sized for a lateral flow matrix; a bottom web attachable to the top web; and a sealing layer allocated on the top web configured to prevent contamination of the lateral flow matrix; wherein the lateral flow matrix includes a sample port. The method of manufacturing includes a series of die cutting mechanisms applied to various webs of material unified via a lamination process.

### CROSS-REFERENCE TO RELATED APPLICATION

This application claims priority to U.S. Provisional Patent Application No. 63/042,702 filed Jun. 23, 2020, the entirety of which is incorporated by reference.

### FIELD OF THE INVENTION

The present invention relates generally to diagnostic testing devices used for determining presence of an infectious disease, and, more particularly, relates to a device for collecting and testing biological samples for diagnostic purposes in addition to methods of manufacturing.

### BACKGROUND OF THE INVENTION

Due to the recent outbreak of infectious diseases, the ability to obtain test results for infectious diseases in real-time has become vital. In addition, chemicals used in a reaction to detect or measure a substance of interest known as reagents are in limited supply, especially when the reagents pertain to an infectious disease that is deemed novel. Due to this limitation, combating and/or controlling massive outbreaks of uncommon infectious diseases is extremely difficult. In order to effectively begin the process of battling a pandemic, testing is required to detect the presence of the applicable infectious disease One approach is to utilize reverse transcription polymerase chain reactions (RT-PCR) to detect the infectious disease; however this approach includes multiples drawbacks. For example, the RT-PCR approach requires ample laboratory space and specifically trained professionals to render it. Although efficient, this approach is costly, includes a high risk of cross-contamination and requires several days to receive results. Utilizing the RT-PCR approach is counterintuitive to preventing dissemination of a novel infectious disease due to the fact that by the time an infected individual receives positive results, there is a high probability that they have been exposed to other individuals. Particularly, this is applicable to individuals who are asymptomatic in regards to the infectious disease.

Another testing approach is to acquire a biological sample such as blood or saliva from a subject and apply the collected biological sample to a lateral flowing matrix resulting in analyte components being detected in the sample when at least one reagent is provided. A common method to rendering this testing approach is by immunoassays, which are configured to provide rapid protocols for untrained users. Immunoassays use a combination of an antibody or antibody-like molecule to capture the molecule of interest. However, this approach includes issues as well. For example in some cases, an antibody or antibody pair may not exist which increases the probability of false results. In addition, the range of analytes and antigens that can be detected with immunoassays is rather limited resulting in an enzymatic modification rendering the immunoassay inefficient. Most importantly, the cost of manufacturing immunoassays is rather high due to a plurality of issues. This includes antibody costs, reagent usage, and lack of preventative measures to account for the high probability of contamination of immunoassays during the manufacturing process.

What is needed is a rapid and efficient diagnostic testing device that circumvents the aforementioned issues, and that can be manufactured in an economically feasible manner while not being subjected to a high probability of contamination.

### SUMMARY OF THE INVENTION

The invention provides a diagnostic testing device that overcome the herein aforementioned disadvantages of the heretofore-known devices.

Although the invention is illustrated and described herein as embodied in a diagnostic testing device, it is, nevertheless, not intended to be limited to the details shown because various modifications and structural changes may be made therein without departing from the spirit of the invention and within the scope and range of equivalents of the claims. Additionally, well-known elements of exemplary embodiments of the invention will not be described in detail or will be omitted so as not to obscure the relevant details of the invention.

With the foregoing and other objects in view, there is provided, in accordance with the invention, a diagnostic testing device is provided that includes a sheet of foam; a top web including a recess shaped and sized for a lateral flow matrix; a bottom web attachable to the top web; and a sealing layer allocated on the top web configured to prevent contamination of the lateral flow matrix; wherein the lateral flow matrix includes a sample port.

In accordance with a further feature of the present invention, in some embodiments, the diagnostic testing device is a rapid testing mechanism wherein the bottom web is attachable to the top web by an adhesive allocated on at least one side of the bottom web, and the sheet of foam, top web, and the bottom web cooperate to define a foam-based cassette structure.

In accordance with a further feature of the present invention, in some embodiments, the sheet of foam is a sterile surgical foam configured to be die cut and the sealing layer includes a breathing port configured to allow moisture to evaporate off of the sealing layer. The sealing layer is removable laminated film from the top web resulting in the recess being exposed.

In accordance with a further feature of the present invention, in some embodiments, the lateral flow matrix is configured to provide testing of a plurality of COVID 19 antibodies.

With the foregoing and other objects in view, there is provided, in accordance with the invention, a method of manufacturing a rapid testing mechanism is provided that includes continuously receiving a first roll of material configured to receive a first series of die cuts forming a plurality of top webs each including a die cut recess; continuously receiving a second roll of material wherein the second roll of material includes an adhesive applied to at least one side of the second roll of material; continuously receiving a reel including a plurality of lateral flow matrixes wherein the reel is configured to receive a second series of die cuts forming a plurality of lateral flow assays; vacuum rolling placement of each lateral flow assay of the plurality of lateral flow assays within the die cut recess of the plurality of top webs; uniting the plurality of top webs to the plurality of bottom webs forming a continuous amalgamated structure; and applying a third series of die cuts to the continuous amalgamated structure forming a plurality of foam-based cassette structures.

Other features that are considered as characteristic for the invention are set forth in the appended claims. As required, detailed embodiments of the present invention are disclosed herein; however, it is to be understood that the disclosed embodiments are merely exemplary of the invention, which can be embodied in various forms. Therefore, specific structural and functional details disclosed herein are not to be interpreted as limiting, but merely as a basis for the claims and as a representative basis for teaching one of ordinary skill in the art to variously employ the present invention in virtually any appropriately detailed structure. Further, the terms and phrases used herein are not intended to be limiting; but rather, to provide an understandable description of the invention. While the specification concludes with claims defining the features of the invention that are regarded as novel, it is believed that the invention will be better understood from a consideration of the following description in conjunction with the drawing figures, in which like reference numerals are carried forward. The figures of the drawings are not drawn to scale.

Before the present invention is disclosed and described, it is to be understood that the terminology used herein is for the purpose of describing particular embodiments only and is not intended to be limiting. The terms “a” or “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The term “coupled,” as used herein, is defined as connected, although not necessarily directly, and not necessarily mechanically. The term “providing” is defined herein in its broadest sense, e.g., bringing/coming into physical existence, making available, and/or supplying to someone or something, in whole or in multiple parts at once or over a period of time.

As used herein, the terms “about” or “approximately” apply to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and explain various principles and advantages all in accordance with the present invention.

FIG. 1 is a perspective view of a diagnostic testing device with an enclosed top-web, according to an exemplary embodiment;

FIG. 2 is a perspective view of the diagnostic testing device of FIG. 1 with an exposed top-web, according to an exemplary embodiment;

FIG. 3 is an exploded view of the diagnostic testing device of FIG. 1, according to an exemplary embodiment;

FIG. 4 is an illustration of a first step of a method of manufacturing the diagnostic testing device; according to an exemplary embodiment;

FIG. 5 is an illustration of a second step of the method of manufacturing the diagnostic testing device; according to an exemplary embodiment;

FIG. 6 is an illustration of a third step of the method of manufacturing the diagnostic testing device; according to an exemplary embodiment;

FIG. 7 is an illustration of a fourth step of the method of manufacturing the diagnostic testing device; according to an exemplary embodiment;

FIG. 8 is an illustration of a reel of lateral flow matrixes used in the method of manufacturing the diagnostic testing device; according to an exemplary embodiment;

FIG. 9 is a diagram of a vacuum pressing technique used in the method of manufacturing the diagnostic testing device; according to an exemplary embodiment;

FIG. 10 is a diagram of a dual-stage die cutting technique for a plurality of webs used in the method of manufacturing the diagnostic testing device; according to an exemplary embodiment;

FIG. 11 is a diagram of a dual-stage die cutting technique for a the reel of FIG. 8 used in the method of manufacturing the diagnostic testing device; according to an exemplary embodiment;

FIG. 12 is a block diagram depicting a method of manufacturing the diagnostic testing device, according to an exemplary embodiment;

### DETAILED DESCRIPTION

While the specification concludes with claims defining the features of the invention that are regarded as novel, it is believed that the invention will be better understood from a consideration of the following description in conjunction with the drawing figures, in which like reference numerals are carried forward. It is to be understood that the disclosed embodiments are merely exemplary of the invention, which can be embodied in various forms.

The present invention provides a novel and efficient testing diagnostic device and method manufacturing said testing diagnostic device configured to include a sheet of foam including a recess shaped and sized for a lateral flow matrix; a top web; a bottom web attachable to the top web; and a sealing layer allocated on the top web configured to prevent contamination of the lateral flow matrix; wherein the lateral flow matrix includes a sample port. The novel and efficient method of manufacturing provides continuously receiving a first roll of material configured to receive a first series of die cuts forming a plurality of top webs each including a die cut recess; continuously receiving a second roll of material wherein the second roll of material includes an adhesive applied to at least one side of the second roll of material; continuously receiving a reel including a plurality of lateral flow matrixes wherein the reel is configured to receive a second series of die cuts forming a plurality of lateral flow assays; vacuum rolling placement of each lateral flow assay of the plurality of lateral flow assays within the die cut recess of the plurality of top webs; uniting the plurality of top webs to the plurality of bottom webs forming a continuous amalgamated structure; and applying a third series of die cuts to the continuous amalgamated structure forming a plurality of foam-based cassette structures. The aforementioned method of manufacturing provides a cost preventative mechanism configured to generate the rapid testing diagnostic device in a manner that circumvents the rapid testing diagnostic device being compromised.

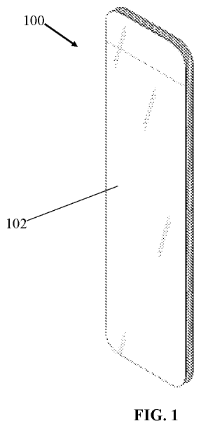

Referring now to FIG. 1, a diagnostic testing device or rapid testing mechanism 100 ready for use by a subject, according to an exemplary embodiment. It is to be understood that as presented the diagnostic testing device is configured to include features known to those of ordinary skill in the art including but not limited to testing strips, qualitative and quantitative indicators for analytes, electrodes, substrates (polymers, polyimides, etc.), or any other applicable chemistry-based component. In some embodiments, diagnostic testing device 100 includes a protective covering and/or sealing layer 102 configured to protect the interior components of device 100 and more importantly to prevent contamination of components and/or sub-components, such as the lateral flow assay, included within device 100. It is to be understood that protective covering 102 is configured to be removable via peeling or any other applicable action provided by the subject configured to expose interior components and/or sub-components of protective covering 102. In some embodiments, protective covering 102 is configured to be laminated. In some embodiments, protective covering 102 includes graphics, designs, insignia (source-based and/or indicating instruction of use), or any other applicable markings. For example, protective covering 102 may include one or more arrows indicating the direction in which the applicable action may be applied to remove protective covering 102.

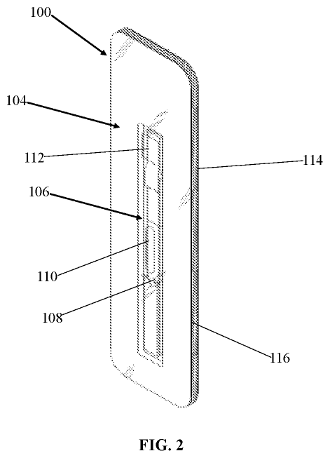

Referring now to FIG. 2, diagnostic testing device 100 is presented with protective covering 102 completely removed, according to an exemplary embodiment. However, in some embodiments, protective covering 102 does not need to be completely removed in order to expose interior components of diagnostic testing device 100. It is to be understood that removal of protective covering 102 exposes a top web 104 designed and configured to include a recess 106. Recess 106 may serve as a housing and/or seating tray for a lateral flow matrix 108 or assay. In a preferred embodiment, lateral flow matrix 108 is designed and configured to assist with detection and diagnosis of COVID 19 and/or indicators of COVID 19. In some embodiments, lateral flow matrix 108 may include one or more openings 110 and 112 configured to serve as sites for reception of biological samples of the subject, sample ports, ventilation windows/slit, breathing port, or any other applicable aperture known to those ordinary skill in the art. In the embodiment where openings 110 and/or 112 is serving has a breathing port, to allow moisture to evaporate off of top web 104 or protective covering 102 in the configuration where protective covering 102 is partially attached. In a preferred embodiment, biological samples received at one of openings 110 and 112 allows traversing of a lateral path flow via lateral flow matrix 108. In some embodiments, openings 110 and 112 may be applied via kiss-cut machinery, die-cut machinery, and/or laser perforation subject to the applicable configuration. In some embodiments, diagnostic testing device 100 may include the dimensions of 4 mm; however, it is within the spirit and scope of this disclosure that diagnostic testing device 100 may support various shapes, dimensions, and orientations as long as the aforementioned do not serve as mechanisms to impact or compromise the efficiency and functionality of lateral flow matrix 108. In some embodiments, diagnostic testing device 100 further includes a bottom web 114 in which top web 104 and bottom web 114 cooperate to assist define a space and/or area designated for a foam sheet 116. In some embodiments, foam sheet 116 includes recess 106 allowing top web 104 to serve as protective or securing layer for lateral flow matrix 108.

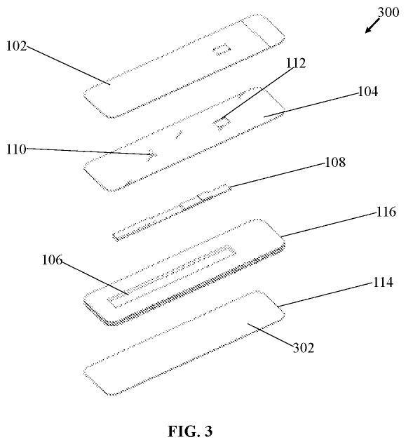

Referring to FIG. 3, an exploded view 300 of the diagnostic testing device 100 and its components are provided, according to an exemplary embodiment. As depicted, top web 104 serves as a protective or sealing mechanism for recess 106 and lateral flow matrix 108, in which foam sheet 116 is composed of a surgical foam. In some embodiments, openings 110 and/or 112 are allocated directly on top web 104. In some embodiments, bottom web 114 includes at least one side including an adhesive and/or affixing material 302 including but not limited to applicable glues, pastes, velcro, or any other adhesive or affixing material and/or substances known to those of ordinary skill in the art. It is to be understood that the purpose of adhesive 302 is to ensure attachment of bottom web 114 to top web 104 and/or foam sheet 116.

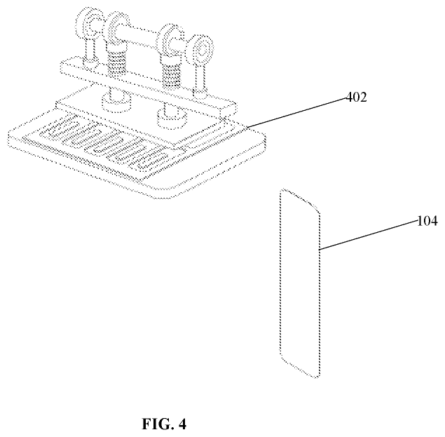

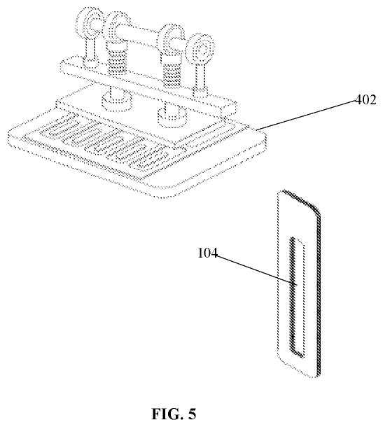

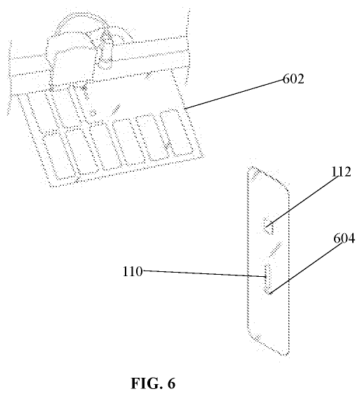

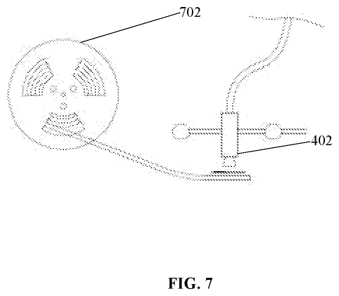

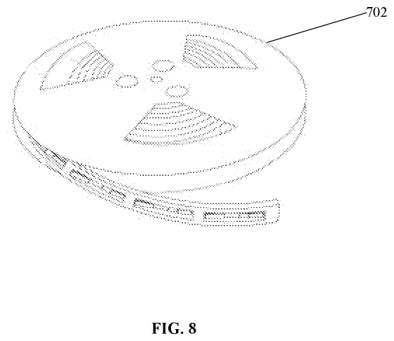

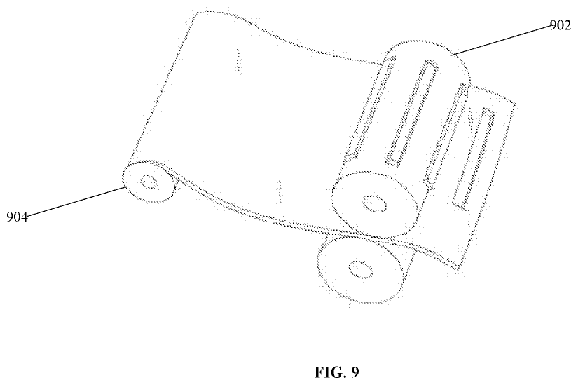

Referring now to FIGS. 4-9, exemplary machinery configured to be integrated into a method of manufacturing diagnostic testing device 100 is illustrated, according to an exemplary embodiment. It is to be understood that the exemplary machinery provided herein may be configured for the use of mass custom production and manufacturing in which the methods disclosed herein may be completed in a centralized method of manufacturing or in divided components and subcomponents working in unison to manufacture diagnostic testing device 100. As described herein, a die-cutting machine 402, a kiss-cutting machine 602, a reel containing a plurality of lateral flow matrixes 702, a vacuum press machine 902, and a roll of film/material 904 is provided. It is to be understood that additional machinery and assisting components may be included in the method of manufacturing including but not guillotine machinery, laminating machinery, shafts, feeds, spindles, carriages, and any other machinery and applicable components known to those of ordinary skill in the art. It is to be understood that top web 104, bottom web 114, and foam sheet 116 are derived from a respective source of each presented in a manner such as roll 904 allowing the materials used to make the aforementioned to be continuously feed from roll 904 to die-cutting machine 402 and/or any other applicable machinery. In some embodiments, reel 702 is continuously fed into die-cutting machine 402 to generate individual lateral flow matrixes (as depicted in FIG. 7) configured to be positioned into recess 106 by vacuum press machine 902 via naturally operation of an assembly-line type configuration. In some embodiments, robotic devices may be used in replace of vacuum press machine 902 in order to properly place and position the lateral flow matrixes within recess 106. In some embodiments, the die cutting of top web 104, bottom web 114, and foam sheet 116, respectively, may be accomplished by a singular or compilation of die-cutting machines 402 working together.

In some embodiments, kiss-cutting machine 602 may be utilized to provide openings 110 and 112 either on top web 104 or a protective covering associated with top web 104 that seeks to prevent contamination of lateral flow matrix 108. In addition, kiss-cutting machine 602 may apply a breathing port 604 designed and configured to allow moisture to evaporate off of the sealing layer at least one of protective covering 102, top web 104, and other applicable sealing or protective layer. Alternatives to kiss-cutting machine 602 include laser perforation mechanisms or any other applicable mechanisms for sanitary incisions or inscribing known to those of ordinary skill in the art. In some embodiment, software and/or centralized software platforms may be integrated and/or associated with the aforementioned mechanisms configured to be used in order to render diagnostic testing device 100. For example, software and/or centralized software platforms may be configured to actuate one or more of die-cutting machine 402, kiss-cutting machine 602, or any of the aforementioned machinery during any point in time of the method of manufacturing.

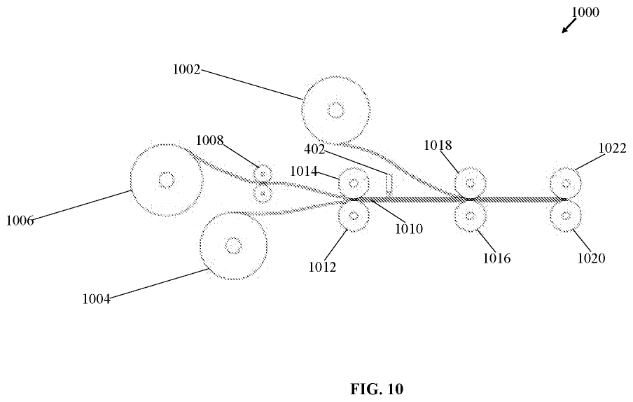

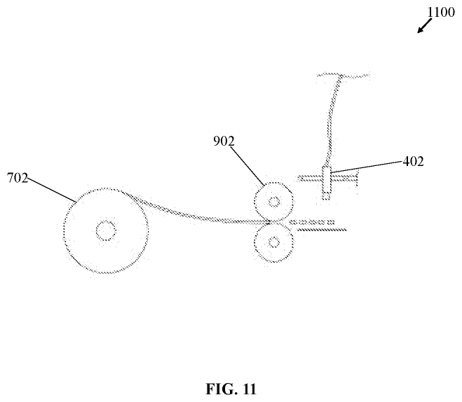

Referring now to FIGS. 10-11, an environment 1000 for a method of manufacturing for diagnostic testing device 100 to be performed in depicted, according to an exemplary embodiment. In some embodiments, environment 1000 includes a first roll of top web material 1002, a second roll of bottom web material 1004, and a third roll of surgical foam material 1006. It is to be understood that rolls 1002-1006 are configured to be continuously feed into an assembly line 1010 which is configured to convey and combine the respective materials of each roll in order to generate diagnostic testing device 100. In some embodiments, a foam die cutter machine 1008 is configured to apply die cuts specifically to materially being continuously received from third roll of foam material 1006. In some embodiments, the applied die cuts are applied by foam die cutter machine 1008 prior to the foam material coming into contact with first roll of top web material 1002 or second roll of bottom web material 1004. In some embodiments, lamination mechanisms 1012-1022 may be used to laminate and unify materials 1002, 1004, and 1006 at various points in the method manufacture. In some embodiments, once material 1006 comes into contact with material 1004 the two materials are combined and laminated by lamination mechanisms 1012 and 1014. In some embodiments, the combination of material 1004 and material 1006 are introduced to material 1002 in which lamination unit 1018 laminates and combines the three materials in preparation for the materials to finally laminated by lamination mechanism 1022 and cut be a final die cutter mechanism 1020.

In some embodiments, while the aforementioned combining and laminating is occurring, a sub-component environment 1100 as depicted in FIG. 11 provides an environment for reel 702 to be continuously die cut and laminated if applicable. In some embodiments, reel 702 is being continuously fed to vacuum press machine 902 in order to ensure proper placement of lateral flow matrix 108 derived from reel 702 into recess 106, in which lateral flow matrix 108 may be laminated and/or die cut depending on the configuration. It is to be understood that sub-component environment 1100 is configured to be integrated into environment 1000 in which assembly line 1010 seamlessly integrates lateral flow matrix 108 into recess 106 prior to the combination of materials 1002, 1004, and 1006 being unified and cut.

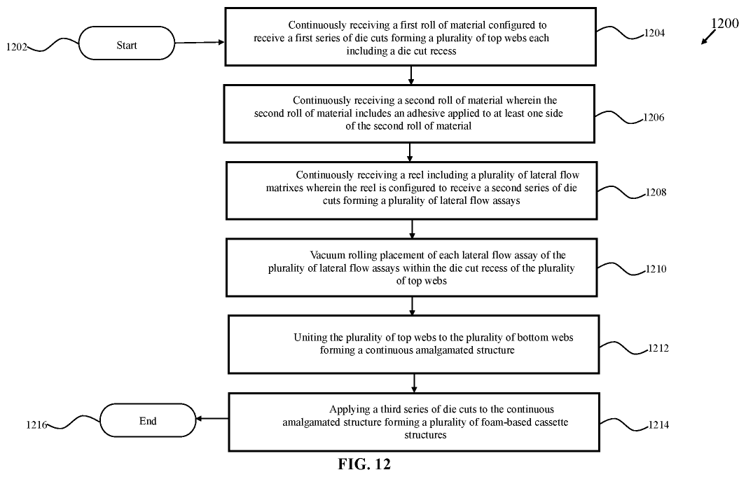

Referring now to FIG. 12, a method of manufacturing 1200 for diagnostic testing device 100 is depicted, accordingly to an exemplary embodiment. The method starts at step 1202, in which materials 1002, 1004, and 1006 prepared within their respective positions in order to be continuously received by assembly line 1010. At step 1204, assembly line 1010 continuously receives materials 1002 allowing the top webs to be prepared via the die cutting and lamination process. In some embodiments, material 1002 includes a label prior to the material being provided to assembly line 1010. At step 1206, assembly line 1010 continuously receives materials 1004 allowing the bottom webs to be prepared via the die cutting and lamination process. In some embodiments, material 1004 includes an adhesive allocated on at least one side prior to the material being provided to assembly line 1010. At step 1208, reel 702 is continuously fed one of the aforementioned die cutting machines in order to form the plurality of assays. At step 1210, the plurality of assays are fed into vacuum press machine 902 allowing the assays to be placed into appropriate position which is ultimately recess 106. In some embodiments, the vacuum rolling step is performed before reel 702 is sliced subject to the applicable configuration. At step 1212, materials 1002 and 1004 are united via lamination applied by at least one of the aforementioned laminating mechanisms allowing the top webs and bottom webs to form a continuous amalgamated structure. It is to be understood that any appropriate time prior to step 1214 material 1006 is die cut and introduced into assembly line 1010. At step 1214, a series of die cuts are applied to the resulting continuous amalgamated structure, including material 1006 as well, resulting in the production of a plurality of diagnostic testing device 100.

The claims appended hereto are meant to cover all modifications and changes within the scope and spirit of the present invention.

What is claimed is:

1. A rapid testing mechanism comprising:
a sheet of foam;
a top web including a recess shaped and sized for a lateral flow matrix;
a bottom web attachable to the top web; and
a sealing layer allocated on the top web configured to prevent contamination of the lateral flow matrix;
wherein the lateral flow matrix includes a sample port.

2. The rapid testing mechanism of claim 1, wherein the bottom web is attachable to at least one of the sheet of foam or the top web by an adhesive allocated on at least one side of the bottom web.

3. The rapid testing mechanism of claim 1, wherein the sheet of foam, top web, and the bottom web cooperate to define a foam-based cassette structure.

4. The rapid testing mechanism of claim 1, wherein the sheet of foam is a sterile surgical foam configured to be die cut.

5. The rapid testing mechanism of claim 1, wherein the sealing layer includes a breathing port configured to allow moisture to evaporate off of the sealing layer.

6. The rapid testing mechanism of claim 1, wherein when the sealing layer is removed from the top web the recess is exposed.

7. The rapid testing mechanism of claim 1, wherein the rapid testing mechanism is 4 mm.

8. The rapid testing mechanism of claim 1, wherein the sealing layer is a laminated film.

9. The rapid testing mechanism of claim 1, wherein the sample port is configured to receive a biological sample which traverses the lateral flow path via the lateral flow matrix.

10. The rapid testing mechanism of claim 1, wherein the lateral flow matrix is configured to provide testing of a plurality of COVID 19 antibodies.

11. A method of manufacturing a rapid testing mechanism comprising:
continuously receiving a first roll of material configured to receive a first series of die cuts forming a plurality of top webs each including a die cut recess;
continuously receiving a second roll of material wherein the second roll of material includes an adhesive applied to at least one side of the second roll of material;
continuously receiving a reel including a plurality of lateral flow matrixes wherein the reel is configured to receive a second series of die cuts forming a plurality of lateral flow assays;
vacuum rolling placement of each lateral flow assay of the plurality of lateral flow assays within the die cut recess of the plurality of top webs;
uniting the plurality of top webs to the plurality of bottom webs forming a continuous amalgamated structure; and
applying a third series of die cuts to the continuous amalgamated structure forming a plurality of foam-based cassette structures.

12. The method of manufacturing of claim 11, wherein continuously receiving the second roll of material includes continuously receiving a third roll of material configured to receive a series of pre-combination die cuts.

13. The method of manufacturing of claim 12, further comprising combining the second roll of material and the third roll of material via a first lamination process.

14. The method of manufacturing of claim 12, wherein the third roll of material is composed of surgical foam.

15. The method of manufacturing of claim 11, wherein the plurality of top webs includes a pre-attached label.

16. The method of manufacturing claim of 11, wherein vacuum rolling placement includes robotic-based placement of each lateral flow assay of the plurality of lateral flow assays.

17. The method of manufacturing claim of 11, wherein uniting the plurality of top webs to the plurality of bottom webs is accomplished by a second lamination process.

18. The method of manufacturing claim of 12, wherein uniting the plurality of top webs to the plurality of bottom webs includes integrating the third roll of material.

19. The method of manufacturing claim of 11, wherein the plurality of lateral flow matrixes are configured to provide testing of a plurality of COVID 19 antibodies.
